(19) United States
(12) Patent Application Publication
(10) Pub. No.: US 2025/0149120 A1
(43) Pub. Date: May 8, 2025

(54) SYSTEMS AND METHODS FOR IN-SILICO BIOPANNING

(71) Applicant: Clara Foods Co., Daly City, CA (US)

(72) Inventors: A Samuel POTTINGER, Daly City, CA (US); Dane Mathias JACOBSON, Daly City, CA (US); Ranjan PATNAIK, Daly City, CA (US); Zachary FRIAR, Daly City, CA (US); Joel Andrew KREPS, Daly City, CA (US); Varsha GOPALAKRISHNAN, Daly City, CA (US)

(73) Assignee: Clara Foods Co., Daly City, CA (US)

(21) Appl. No.: 18/942,210

(22) Filed: Nov. 8, 2024

Related U.S. Application Data
(63) Continuation of application No. PCT/US2023/021772, filed on May 10, 2023.
(60) Provisional application No. 63/340,860, filed on May 11, 2022.

(51) Int. Cl. G16B 35/00; C12N 15/10; G16B 40/20

(52) U.S. Cl. CPC G16B 35/00; C12N 15/1089; G16B 40/20

(57) ABSTRACT

Disclosed is a method for identifying a subset of high-expressing proteins. The method comprises (a) generating a distance matrix relating a plurality of amino acid sequences of a training set of amino acid sequences from a plurality of training proteins. The method also comprises (b) performing dimensionality reduction on a set of the plurality of amino acid sequence distances of the distance matrix to produce a set of features. The method also comprises (c) generating one or more trained models by training a set of classifiers or regressors on the set of features. The method also comprises (d) using the one or more trained models, analyzing a test set of amino acid sequences from the plurality of proteins, to identify whether the subset of proteins within the plurality of proteins comprises high-expressing proteins.

### CROSS-REFERENCE

This application is a continuation of International Application No. PCT/US2023/021772, filed May 10, 2023, which claims priority to U.S. Provisional Application No. 63/340,860, filed May 11, 2022; the contents of each of which is incorporated by reference herein in its entirety.

### BACKGROUND

Recombinant protein supplements and related food products may require large amounts of recombinant proteins to be tested to determine which proteins would have desired features for use in these supplements or food products. A standard way to generate recombinant proteins is to encode the target protein into DNA and introduce the DNA via expression vector into a host cell, then use the cell's protein synthesis machinery to produce many copies of the recombinant protein. Some proteins are more easily produced in cells in large numbers. It is advantageous to distinguish these “high-expression” or “high-expressing” proteins from others that do not express as well.

### SUMMARY

Surveying different types of proteins in a lab to determine which are high-expression or high-expressing may be costly and inefficient. There is a need for a faster, more efficient process for surveying large numbers of proteins to determine whether they will recombinantly express well in cells. The disclosed system provides an in-silico method using machine learning to predict whether a recombinant protein will be high-expressing.

In an aspect, a method for identifying a subset of high-expressing proteins suitable for use in a recombinant expression system that generates recombinant proteins within a plurality of proteins is disclosed. The method may include (a) generating a distance matrix relating a plurality of amino acid sequences of a training set of amino acid sequences from a plurality of training proteins. The distance matrix comprises a plurality of amino acid sequence measures of similarity. The method may next include (b) performing dimensionality reduction on a set of the plurality of amino acid sequence distances of the distance matrix to produce a set of features. The method may next comprise (c) generating one or more trained models by training a set of classifiers or regressors on the set of features. Finally, the method may comprise (d) using the one or more trained models, analyzing a test set of amino acid sequences from the plurality of proteins, to identify whether the subset of proteins within the plurality of proteins comprises high-expressing proteins. Analyzing the test set of amino acid sequences comprises: (i) repeating steps (a) and (b) for the test set of amino acid sequences to produce a set of test features; and
(ii) processing the set of features with the one or more trained models to predict an expressivity of one or more proteins corresponding with one or more amino acid sequences from the test set of amino acid sequences.

In some embodiments, an amino acid sequence corresponds to a protein or to a fragment of a protein.

In some embodiments, the plurality of proteins includes none of the plurality of training proteins.

In some embodiments, the distance matrix is created at least in part by calculating a Levenshtein distance between at least one pair of the plurality of amino acid sequences.

In some embodiments, the distance matrix is calculated at least in part using a Floyd-Warshall algorithm.

In some embodiments, the dimensionality reduction is principal component analysis (PCA) or multidimensional scaling (MDS).

In some embodiments, the multidimensional scaling uses from about 20 to about 40 components.

In some embodiments, the set of classifiers comprises at least one of a logistic regression, a decision tree, gradient boosted trees, a random forest model, neural network or Adaboost.

In some embodiments, the set of features comprises no more than 20 features.

In some embodiments, the set of features comprises from about 10 to about 20 features.

In some embodiments, a protein of the set of high-expressing proteins is expressed in a cell.

In some embodiments, the cell is a recombinant cell.

In some embodiments, the recombinant cell is a microbial cell.

In some embodiments, the microbial cell is from a microbial organism selected from the group consisting of a Komagataella species, a Saccharomyces species, a Trichoderma species, a Pseudomonas species, an Aspergillus species, and an E. coli species.

In some embodiments, the plurality of proteins includes one or more animal or egg proteins.

In some embodiments, the one or more animal or egg proteins are selected from ovalbumin (OVA), ovomucoid (OVD), ovotransferrin, lysozyme proteins, ovomucin, ovoglobulin G2, ovoglobulin G3, ovoinhibitor, ovoglycoprotein, flavoprotein, ovomacroglobulin, ovostatin, cystatin, avidin, ovalbumin related protein X, and ovalbumin related protein Y, or any combination thereof.

In some embodiments, the one or more animal or egg proteins are naturally expressed by poultry, fowl, waterfowl, game bird, chicken, quail, turkey, turkey vulture, hummingbird, duck, ostrich, goose, gull, guineafowl, pheasant, emu, crocodile, owl, finch, pigeon, penguin, or any combination thereof. Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a system for predicting high-expressivity proteins in-silico, in accordance with some embodiments;

FIG. 2 illustrates a pre-processing subsystem, in accordance with some embodiments;

FIG. 3 illustrates a machine learning subsystem, in accordance with some embodiments;

FIG. 4 illustrates a process flow diagram for training a machine learning model;

FIG. 5 illustrates a process flow diagram showing prediction of expressivity levels of various proteins;

FIG. 6 illustrates a performance comparison of the system between multidimensional scaling and principal component analysis;

FIG. 7 illustrates results of an in-silico biopanning experiment; and

FIG. 8 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### Overview

Disclosed is a system that uses machine learning for virtual biopanning- or analyzing large numbers of proteins to determine whether they will express well when recombinantly introduced into cells (i.e., are “high-expression” or “high-expressing” proteins). The system may use information obtained from in vivo biopanning to train the machine learning models, and then may test the model using lab results, synthetic results, or both.

First, the system may train the machine learning models used to predict protein expressivity. The system obtains data (e.g., from researchers) comprising amino acid sequences of various proteins and expressivity levels of these proteins. The system may pre-process this data for machine learning analysis, by converting the sequence data into a matrix representation and applying one or more dimensionality reduction techniques. Then, the system may apply this pre-processed data to one or more machine learning algorithms to train them.

Next, the system may obtain additional data comprising protein sequences that have not been used to train the machine learning algorithm. This data may be obtained from a lab or generated or may be a hybrid of both types of data. The system may pre-process the data using a substantially similar process to the one used to pre-process the training data. The system may then implement the machine learning algorithms to produce predictions of expressivity for each of the input proteins.

### Particular Implementations

A method for identifying a subset of high-expressing proteins suitable for use in a recombinant expression system that generates recombinant proteins within a plurality of proteins is disclosed. A protein may be verified as high-expressing using an expert review of a gel image or by using titers. A threshold dividing high and low expression in proteins may be based on “expert review” of gel images on a Likert scale, where an integer score of 0 or 1 is low and 2 or 3 is high. These gel images may show how well a protein was expressed and are reviewed visually. However, one may also use amino acid fingerprinting to determine high and low expression.

The method may comprise generating a distance matrix relating a plurality of amino acid sequences of a training set of amino acid sequences from a plurality of training proteins. The distance matrix may comprise string metric values relating each of the amino acid sequences in the set of training proteins to every other amino acid sequence in the training set.

The string metric used may be an edit distance (Levenshtein distance). For example, the method may comprise calculating a Levenshtein distance by computing a number of single-character edits (insertions, deletions, or substitutions) required to change one amino acid sequence into another amino acid sequence. For example, amino acid sequences AAAAAN and AAAAN may have a Levenshtein distance of 1.

The distance matrix may additionally use other string metrics, including Damerau-Levenshtein distance, Sorensen-Dice coefficient, block distance (L1 distance or city block distance), Hamming distance, Jaro-Winkler distance, simple matching coefficient, Jaccard similarity, Jaccard coefficient, Tanimoto coefficient, Tversky index, overlap coefficient, variational distance, Hellinger distance or Bhattacharyya distance, Information radius, skew divergence confusion probability, Tau metric, Kullback-Leibler divergence, Fellegi and Sunters metric, Maximal matches, or grammar-based distance.

The distance matrix may comprise a plurality of amino acid sequence measures of similarity. The distance matrix may comprise, for example, rows and columns, where a (row, column) location of the distance matrix indicates a string metric (e.g., Levenshtein distance or another type of character-based edit distance) between a protein corresponding to the ordinal row and a protein corresponding to the ordinal column. For example, for a protein labeled with A1 and a protein A2, the location (A1, A2) could indicate the Levenshtein distance between proteins A1 and A2.

In some embodiments, the string metrics are placed into a graph. The graph may include amino acid sequences as nodes, with the edges of the graph being magnitude measures of difference between the nodes and all other nodes (e.g., Levenshtein distances). In some embodiments, the distance matrix is generated at least in part using a Floyd-Warshall algorithm. The Floyd-Warshall algorithm may used in cases where a fully connected (or complete) graph of protein sequences exists with edges indicating string distance metrics between sequences. The Floyd-Warshall algorithm may generate shortest path vectors from each amino acid sequence to every other sequence, based on the weights of the edges in the graph (e.g., the magnitude measures of distance). From the shortest path vectors, the system may generate a distance matrix by summing the weights in the shortest path vectors and populating the matrix with the sums. For example, if the sum of weights in the shortest path from amino acid sequence A1 to amino acid sequence A2 is equal to 4, the locations of the matrix corresponding to (row, column)=(A1, A2) and (row, column)=(A2, A1) could have a value equal to 4. Thus, the Floyd-Warshall algorithm may generate the distance matrix by filtering for protein-to-protein edit distances.

The method may next comprise performing dimensionality reduction on a set of amino acid sequence distances of the distance matrix to produce a set of features for machine learning. The dimensionality reduction techniques may be unsupervised techniques such as principal component analysis (PCA), multidimensional scaling (MDS), non-negative matrix factorization (NFM), kernel PCA, graph-based kernel PCA, use of an autoencoder, t-distributed stochastic neighbor embedding (t-SNE), or uniform manifold approximation and projection (UMAP). The dimensionality reduction techniques may be unsupervised techniques.

In some embodiments, the multidimensional scaling uses greater than about 10, greater than about 15, greater than about 20, greater than about 30, greater than about 40, or greater than about 50 components. In some embodiments, the multidimensional scaling uses less than about 15, less than about 20, less than about 30, less than about 40, less than about 50, or less than about 60 components. In some embodiments, the multidimensional scaling uses from about 10 to 20, from about 20 to 30, from about 30 to 40, from about 40 to 50, or from about 50 to 60 components.

The method may generate one or more trained models by training a set of classifiers or regressors on the set of features. The classifiers may be binary or multiclass classifiers. Classifiers may be used to determine whether proteins are low or high-expressing. Regressors may be used to predict specific expression values of proteins. The classifier may use an algorithm such as a logistic regression, decision tree, gradient boosted trees, Adaboost, random forest, Naïve Bayes, k-nearest neighbors, perceptron, or support vector machine (SVM). In some embodiments, the method uses a neural network.

The method may analyze a test set of amino acid sequences. The test set of amino acid sequences may include amino acid sequences that were not trained on. The method may analyze these sequences to determine whether these proteins are high-expressing proteins. Analysis of the test set may be pursued using the same or similar techniques for training the model. Prior to analysis with a machine learning algorithm, the system may generate a distance matrix relating the plurality of amino acid sequences. Then, the system may implement dimensionality reduction on the data in the distance matrix to generate a set of features. The system may then implement the trained machine learning algorithm on the features to generate predictions of expressivity for proteins comprising the test set of amino acid sequences.

An amino acid sequence may correspond to a protein or a fragment of a protein. For example, the amino acid sequences in the training or test sets may be peptides or polypeptides.

In some embodiments, the proteins are expressed in a cell. In some embodiments, the cell is a recombinant cell. In some embodiments, the recombinant cell is a microbial cell.

The microbial cells may be from organisms from a Komagataella species, a Saccharomyces species, a Trichoderma species, a Pseudomonas species, an Aspergillus species, or an E. coli species.

In some cases, the recombinant cell may be a methanotroph. Among methanotrophs, Komagataella pastoris and Komagataella phaffii are preferable (also known as Pichia pastoris). Examples of strains in the Komagataella genus include Pichia pastoris strains. Examples can include NRRL Y-11430, BG08, BG10, NRRL Y-11430 GS115 (NRRL Y-15851), GS190 (NRRL Y-18014), PPF1 (NRRL Y 18017), PPY120OH, YGC4, and strains derived therefrom. Other examples of P. pastoris strains that may be used as host cells include but are not limited to CBS7435 (NRRL Y-11430), CBS704 (DSMZ 70382) or derivatives thereof. Other examples of methanol-utilizing yeast include yeasts belonging to Ogataea (Ogataea polymorpha), Candida (Candida boidinii), Torulopsis (Torulopsis) or Komagataella.

Further examples of suitable host cell organisms include but are not limited to eukaryotic cells such as: Arxula spp., Arxula adeninivorans, Kluyveromyces spp., Kluyveromyces lactis, Komagataella spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, Aspergillus oryzae, Bacillus subtilis, Escherichia coli, Myceliophthora thermophila, Neurospora crassa, Pichia pastoris, Pichia Pastoris “MutS” strain (Graz University of Technology (CBS7435MutS) or Biogrammatics (BG11)), Komagatella phaffi, and Komagatella pastoris.

In some cases, a bacterial host cell such as Lactococcus lactis, Bacillus subtilis or Escherichia coli may be used as the host cells. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Pseudomonas sp., Pseudomonas fluorescens.

The proteins may be animal or egg proteins. The egg proteins may naturally be expressed by poultry, fowl, waterfowl, game bird, chicken, quail, turkey, turkey vulture, hummingbird, duck, ostrich, goose, gull, guineafowl, pheasant, emu, crocodile, owl, finch, pigeon, or penguin eggs.

The egg-proteins may be ovalbumin (OVA), ovomucoid (OVD), ovotransferrin, lysozyme proteins, ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, or any combination thereof.

### System

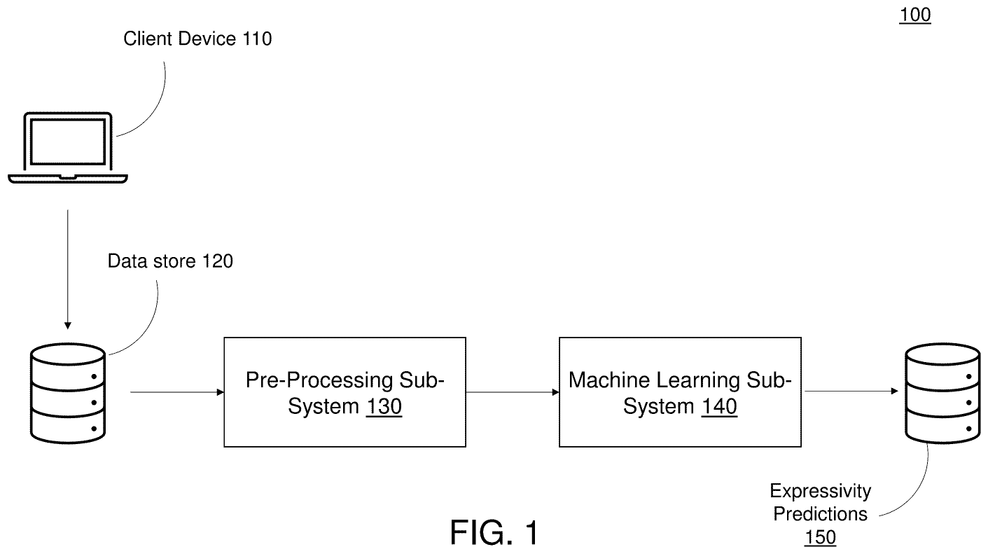

FIG. 1 illustrates a system 100 for predicting high-expressivity proteins in-silico. The system includes a client device 110, a data store 120, a pre-processing sub-system 130, and a machine learning sub-system 140.

The client device 110 may be a computing device used to upload or provide amino acid sequence data to other system components. The client device 110 may be a mobile device. The client device 110 may be a desktop computer, mainframe computer, cellular phone, smartphone, tablet computer, personal digital assistant (PDA), supercomputer, or another type of computer. The client device 110 may be associated with a laboratory where various proteins are tested for their expressivity. Researchers may use the client device 110 to upload the amino acid sequence data along with corresponding expressivity data to other system components.

The client device 110 may also present training or testing results to researchers or other stakeholders. For example, the client device 110 may comprise a desktop or web-based (e.g., browser) application for retrieving and presenting protein expressivity results in a graphical user interface (GUI).

The data store 120 may be a computer storage system configured to store protein information in memory. The protein information may be uploaded or retrieved from the client device 110 or from another server comprising a database of protein information. The protein information may comprise amino acid sequences for various proteins in textual form.

The data store 120 may comprise synthetic or non-synthetic data, or a mixture of both. For example, the data may include results from small-scale biopanning, or testing done to see which proteins have high expression. This data may serve as ground truth data to train a machine learning algorithm.

The pre-processing sub-system 130 prepares the protein data for machine learning analysis. The pre-processing sub-system 130 may include one or more components for creating a graphical representation of the protein information. The pre-processing sub-system 130 may comprise software components for producing string metrics from amino acid sequence data, creating graph and/or matrix representations from the string metrics, and implementing dimensionality reduction. The pre-processing sub-system 130 may also provide other pre-processing functions, such as cleaning the data, de-duplicating the data, or removing noise from the data.

Machine learning sub-system 140 may implement one or more machine learning algorithms on the pre-processed amino acid sequences. The machine learning sub-system 140 may implement one or more machine learning algorithms (e.g., logistic regression or random forests). Machine learning sub-system 140 generates expressivity predictions 150, which may be accessed by client device 110.

The systems herein may be implemented on one server machine or multiple machines, or in a distributed system. In some embodiments, components may transfer data to one another over a network. In some embodiments, the network may be a wireless network. In some embodiments, the system may use a cloud architecture.

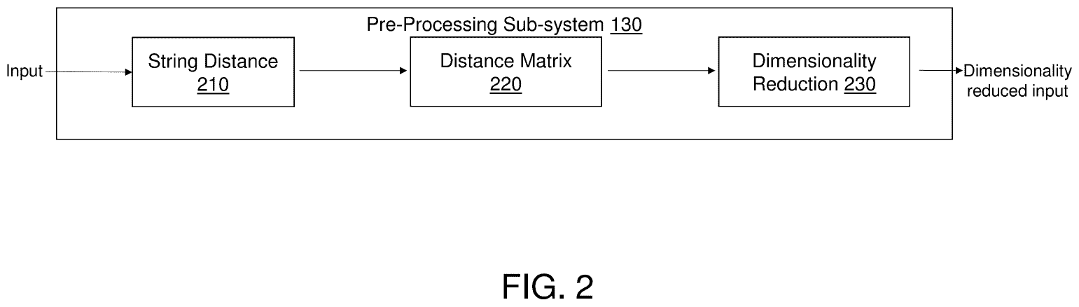

FIG. 2 illustrates pre-processing sub-system 130. The pre-processing sub-system 130 may include a string distance component 210, a distance matrix component 220, and a dimensionality reduction component 230.

The string distance component 210 may generate a graph from string metrics associated with the protein information. In one embodiment, the system may generate Levenshtein distances from each amino acid sequence in the set to every other amino acid sequence in the set. In other embodiments, the system may use additional or alternative string metrics to generate the graph.

The distance matrix component 220 may generate a distance matrix from the graph. In some embodiments, the distance matrix component 220 may generate a distance matrix from the string metrics (e.g., from the Levenshtein distances). In some embodiments, (e.g., where a graph representation of amino acid string metrics is available) the pre-processing sub-system 130 may use a Floyd-Warshall algorithm to determine shortest paths from each protein's amino acid sequence to each of the other protein's amino acid sequences. The distance matrix component 220 may then, from vectors comprising all the shortest paths, generate the distance matrix.

The dimensionality reduction component 230 may perform dimensionality reduction on the data inside the distance matrix. The dimensionality reduction component 230 may use a technique such as principal component analysis (PCA), multidimensional scaling (MDS), representation learning, or another method. Principal component analysis may calculate the eigenvectors of the distance matrix and determine which eigenvectors correspond to the largest eigenvalues. Then, the technique may retain a number of eigenvectors corresponding to the largest eigenvalues (e.g., 20 or 40) as the principal components, and discard the rest. These principal components retain the essential features of the proteins in a compressed format. Alternatively, the dimensionality reduction component 230 may use multidimensional scaling to project the distance matrix into a smaller-dimension (e.g., 2D or 3D) coordinate space.

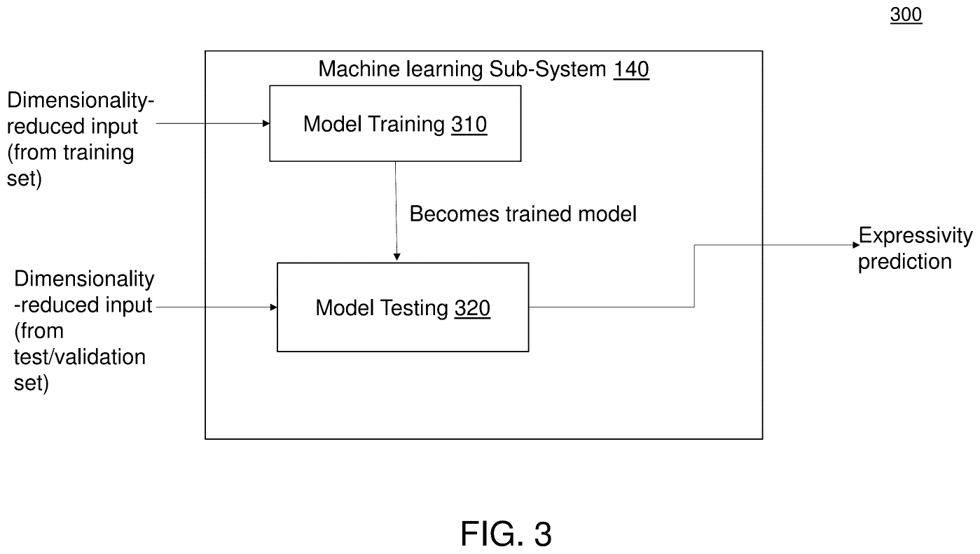

FIG. 3 illustrates machine learning sub-system 140. Machine learning sub-system 140 may include a model training component 310 and a model testing component 320.

The model training component 310 trains the machine learning model used in the machine learning sub-system 140. The machine learning model may comprise one or more machine learning algorithms coupled together in layers. The model training component 310 may receive as input amino acid sequence data retrieved from an in vitro biopanning process. The model training component 310 may implement a set of mathematical operations on the received data to produce an output. The output may be, for example, a score. This score may be compared to a ground truth value, which may be an empirically determined value of expressivity for a particular protein. For some types of machine learning algorithms, the error derived from such a comparison may be iteratively backpropagated through the machine learning algorithm until the score produced by the model is close enough in magnitude to the ground truth value. After training over these several iterations, the machine learning model may be a trained model.

The model testing component 320 uses the trained model to evaluate input data that was not used in training. The input data may comprise amino acid sequences obtained in vivo, in silico, or both. The trained machine learning model's algorithms or algorithm processes the input data to produce predicted values of expressivity for the input proteins.

### Method

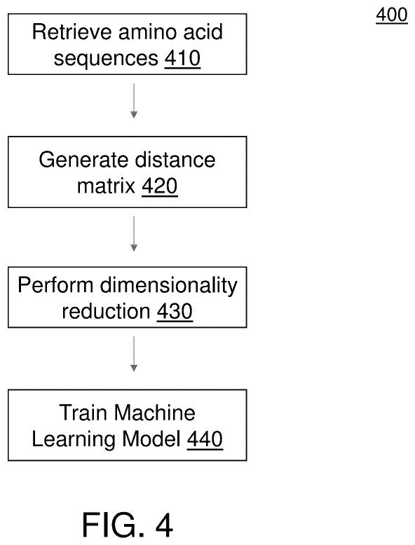

FIG. 4 illustrates a process flow diagram 400 for training a machine learning model.

In a first operation 410, in vivo biopanning is performed to determine ground truth levels of expressivity for various proteins. The amino acid sequences of the proteins along with corresponding expressivity values may be uploaded as a dataset to one or more servers via one or more client devices.

In a second operation 420, a distance matrix is generated from the dataset. The distance matrix may be generated by calculating string metrics for a subset of amino acid sequences from the dataset, and then creating a distance matrix from the string metrics. In some embodiments, the string metrics are Levenshtein distances.

In a third operation 430, the distance matrix is dimensionally reduced. Dimensionality reduction may comprise using a technique such as principal component analysis (PCA) or multidimensional scaling (MDS).

In a fourth operation 440, a machine learning model is trained using data derived from the dimensionally-reduced distance matrix.

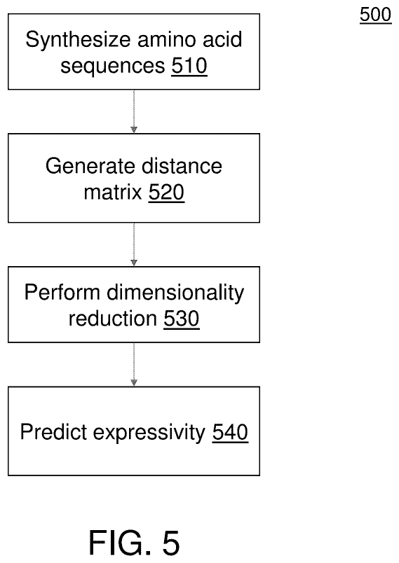

FIG. 5 illustrates a process flow diagram 500 showing prediction of expressivity levels of various proteins.

In a first operation 510, amino acid sequence data is generated. The amino acid sequence data may be generated in silico or may come from in vivo experiments, or both. The sequence data may be placed in a testing or validation dataset.

In a second operation 520, a distance matrix is generated from the dataset. The distance matrix may be a distance matrix generated from string metrics derived from the amino acid sequences.

In a third operation 530, the data in the distance matrix is dimensionally reduced (e.g., using MDS or PCA).

In a fourth operation 540, the trained machine learning algorithm (following operation) is implemented on the dimensionally-reduced data to determine predicted expressivity values for the proteins in the dataset.

### Machine Learning

The methods described herein can comprise computer-implemented methods of supervised or unsupervised learning methods, including support vector machines (SVM), random forests, clustering algorithm (or software module), gradient boosting, logistic regression, and/or decision trees. The machine learning methods as described herein can improve prediction of protein expression as described herein. Machine learning may be used to train a classifier described herein, for example in training a classifier to distinguish between high-expression and low-expression proteins.

Supervised learning algorithms can be algorithms that rely on the use of a set of labeled, paired training data examples to infer the relationship between an input data and output data. Unsupervised learning algorithms can be algorithms used to draw inferences from training data sets to output data. Unsupervised learning algorithms can comprise cluster analysis, which can be used for exploratory data analysis to find hidden patterns or groupings in process data. One example of an unsupervised learning method can comprise principal component analysis. Principal component analysis can comprise reducing the dimensionality of one or more variables. The dimensionality of a given variables can be at least 1, 5, 10, 50, 100, 200, 300, 400, 500, 600, 700, 800, 900, 1000, 1100, 1200 1300, 1400, 1500, 1600, 1700, 1800, or greater. The dimensionality of a given variables can be 1800 or less, 1600 or less, 1500 or less, 1400 or less, 1300 or less, 1200 or less, 1100 or less, 1000 or less, 900 or less, 800 or less, 700 or less, 600 or less, 500 or less, 400 or less, 300 or less, 200 or less, 100 or less, 50 or less, or 10 or less.

The computer-implemented methods can comprise statistical techniques. In some embodiments, statistical techniques can comprise linear regression, classification, resampling methods, subset selection, shrinkage, dimension reduction, nonlinear models, tree-based methods, support vector machines, unsupervised learning, or any combination thereof.

A linear regression can be a method to predict a target variable by fitting a best linear relationship between a dependent and independent variable. The best fit can mean that the sum of all distances between a shape and actual observations at each point is the least. Linear regression can comprise simple linear regression and multiple linear regression. A simple linear regression can use a single independent variable to predict a dependent variable. A multiple linear regression can use more than one independent variable to predict a dependent variable by fitting a best linear relationship.

A classification can be a data mining technique that assigns categories to a collection of data in order to achieve accurate predictions and analysis. Classification techniques can comprise logistic regression and discriminant analysis. Logistic regression can be used when a dependent variable is dichotomous (binary). Logistic regression can be used to discover and describe a relationship between one dependent binary variable and one or more nominal, ordinal, interval or ratio-level independent variables. A resampling can be a method comprising drawing repeated samples from original data samples. A resampling may not involve a utilization of a generic distribution tables to compute approximate probability values. A resampling can generate a unique sampling distribution on a basis of an actual data. In some embodiments, a resampling can use experimental methods, rather than analytical methods, to generate a unique sampling distribution. Resampling techniques can comprise bootstrapping and cross-validation. Bootstrapping can be performed by sampling with replacement from original data and take “not chosen” data points as test cases. Cross validation can be performed by split training data into a plurality of parts.

A subset selection can identify a subset of predictors related to a response. A subset selection can comprise a best-subset selection, forward stepwise selection, backward stepwise selection, hybrid method, or any combination thereof. In some instances, shrinkage fits a model involving all predictors, but estimated coefficients are shrunken towards zero relative to the least squares estimates. This shrinkage can reduce variance. A shrinkage can comprise ridge regression and a lasso. A dimension reduction can reduce a problem of estimating n+1 coefficients to a simpler problem of m+1 coefficients, where m<n. It can be attained by computing n different linear combinations, or projections, of variables. Then these n projections are used as predictors to fit a linear regression model by least squares. Dimension reduction can comprise principal component regression and partial least squares. A principal component regression can be used to derive a low dimensional set of features from a large set of variables. A principal component used in a principal component regression can capture a large amount of variance in data using linear combinations of data in subsequently orthogonal directions. The partial least squares can be a supervised alternative to principal component regression because partial least squares can make use of a response variable in order to identify new features.

A nonlinear regression can be a form of regression analysis in which observational data are modeled by a function which is a nonlinear combination of model parameters and depends on one or more independent variables. A nonlinear regression can comprise a step function, piecewise function, spline, generalized additive model, or any combination thereof.

Tree-based methods can be used for both regression and classification problems. Regression and classification problems can involve stratifying or segmenting the predictor space into a number of simple regions. Tree-based methods can comprise bagging, boosting, random forest, or any combination thereof. Bagging can decrease a variance of prediction by generating additional data for training from the original dataset using combinations with repetitions to produce multistep of the same carnality/size as original data. Boosting can calculate an output using several different models and then average a result using a weighted average approach. A random forest algorithm can draw random bootstrap samples of a training set. Support vector machines can be classification techniques. Support vector machines can comprise finding a hyperplane that best separates two classes of points with the maximum margin. Support vector machines can constrain an optimization problem such that a margin is maximized subject to a constraint that it perfectly classifies data. Unsupervised methods can be methods to draw inferences from datasets comprising input data without labeled responses. Unsupervised methods can comprise clustering, principal component analysis, k-Mean clustering, hierarchical clustering, or any combination thereof.

### Training

A machine learning system as described herein is configured to undergo at least one training phase wherein the machine learning system is trained to carry out one or more tasks including data extraction, data analysis, and generation of output.

In some embodiments of the system, a system component is configured to provide training data, comprising, for example, amino acid sequences of proteins, and corresponding observations of their expressibility, in a training set. In some embodiments, the system utilizes automatic statistical analysis of data to determine which features to extract and/or analyze from a set of amino acid sequences (e.g., a test set of amino acid sequences). In some of these embodiments, the machine learning software module determines which features to extract and/or analyze from a set of amino acid sequences based on the training of the machine learning system.

In some embodiments, a machine learning software module is trained using a data set and a target in a manner that might be described as supervised learning. In these embodiments, the data set is conventionally divided into a training set and a test set and/or a validation set. A target is specified that contains the correct classification of each input value (e.g., high or low expression) in the data set. For example, a set of preprocessed amino acid sequences from many types of proteins is repeatedly presented to the machine learning software module, and for each sample presented during training, the output generated by the machine learning software module is compared with the desired target. The difference between the target and the set of input samples is calculated, and the machine learning system is modified to cause the output to more closely approximate the desired target value. In some embodiments, a back-propagation algorithm is utilized to cause the output to more closely approximate the desired target value. After a large number of training iterations, the machine learning software module output will closely match the desired target for each sample in the input training set. Subsequently, when new input data, not used during training, is presented to the machine learning software module, it may generate an output classification value indicating which of the categories the new sample is most likely to fall into. The machine learning software module is said to be able to “generalize” from its training to new, previously unseen input samples. This feature of a machine learning software module allows it to be used to classify almost any input data which has a mathematically formulatable relationship to the category to which it should be assigned.

In some embodiments of the machine training software module described herein, the machine training software module utilizes a global training model. A global training model is based on the machine training software module having trained on data from sequencing multiple types of proteins and thus, a machine training system that utilizes a global training model is configured to be used to predict expression or expressivity from multiple types of proteins.

In some embodiments of the machine training software module described herein, the machine training software module utilizes a simulated training model. A simulated training model is based on the machine training software module having trained on data from simulated amino acid sequence data from multiple types of proteins. A machine training software module that utilizes a simulated training model is configured to be used on multiple types of proteins.

In some embodiments, the use of training models changes as the availability of amino acid sequence data changes. For instance, a simulated training model may be used if there are insufficient quantities of appropriate amino acid sequence available for training the machine training software module to a desired accuracy. This may be particularly true in the early days of implementation, as few amino acid sequences may be available initially. As additional data becomes available, the training model can change to a global model. In some embodiments, a mixture of training models may be used to train the machine training software module. For example, a simulated and global training model may be used, utilizing a mixture of multiple patients' data and simulated data to meet training data requirements.

Unsupervised learning is used, in some embodiments, to train a machine training software module to use input data such as, for example, amino acid sequence data and output, for example, a prediction of protein expressivity. Unsupervised learning, in some embodiments, includes feature extraction which is performed by the machine learning software module on the input data. Extracted features may be used for visualization, for classification, for subsequent supervised training, and more generally for representing the input for subsequent storage or analysis.

Machine learning software modules that are commonly used for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders. While there are many unsupervised learning models, they all have in common that, for training, they require a training set consisting of biological sequences, without associated labels.

Data that is inputted into the machine learning system may be used, in some embodiments, to construct a hypothesis function to predict protein expression. In some embodiments, a machine learning system is configured to determine if the outcome of the hypothesis function was achieved and based on that analysis determine with respect to the data upon which the hypothesis function was constructed. That is, the outcome tends to either reinforce the hypothesis function with respect to the data upon which the hypothesis function was constructed or contradict the hypothesis function with respect to the data upon which the hypothesis function was constructed. In these embodiments, depending on how close the outcome tends to be to an outcome determined by the hypothesis function, the machine learning algorithm will either adopt, adjust, or abandon the hypothesis function with respect to the data upon which the hypothesis function was constructed. As such, the machine learning algorithm described herein dynamically learns through the training phase what characteristics of an input (e.g., data) are most predictive in determining a level of protein expression.

For example, a machine learning software module is provided with data on which to train so that it, for example, can determine the most salient features of preprocessed amino acid sequence data to operate on. The machine learning software modules described herein train as to how to analyze the sequence data, rather than analyzing the sequence data using pre-defined instructions. As such, the machine learning software modules described herein dynamically learn through training what characteristics of an input signal are most predictive in determining levels of protein expression or protein expressivity.

In some embodiments, training begins when the machine learning system is given amino acid sequence data and asked to predict protein expression for a particular protein. The predicted level of protein expression may then be compared to a true level of protein expression for the particular protein. An optimization technique such as gradient descent and backpropagation is used to update the weights in each layer of the machine learning software module to produce closer agreement between the probability predicted by the machine learning software module, and the actual level of expression. This process is repeated with new preprocessed amino acid sequence data until the accuracy of the network has reached the desired level. Following training with the appropriate amino acid sequence data given above, the machine learning module is able to analyze an amino acid sequence and predict protein expressivity for the protein corresponding with the amino acid sequence.

In general, a machine learning algorithm is trained using a large set of amino acid sequence data or preprocessed amino acid sequence data and/or any features or metrics computed from the above said data with the corresponding ground-truth values. The training phase constructs a transformation function for predicting an expressivity of a protein by using the amino acid sequence data or preprocessed amino acid sequence data and/or any features or metrics computed from the above said data of the unknown patient. The machine learning algorithm dynamically learns through training what characteristics of the input are most predictive of protein expression. A prediction phase uses the constructed and optimized transformation function from the training phase to predict the protein expression for a particular input protein.

### Prediction Phase

Following training, the machine learning algorithm is used to determine, for example, a predicted expressivity of a protein on which the system was trained using the prediction phase. With appropriate training data, the system can identify whether a protein is high-expressing or low-expressing.

The prediction phase uses the constructed and optimized hypothesis function from the training phase to predict the level of expressivity of the protein.

In some embodiments, a probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98% or 99%. In some embodiments, the probability threshold is adjusted if the accuracy, sensitivity or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional amino acid sequence data. In some embodiments, additional amino acid sequences are included in the training data. In some embodiments, additional amino acid sequences can be used to refine the training data set.

### Computer Systems

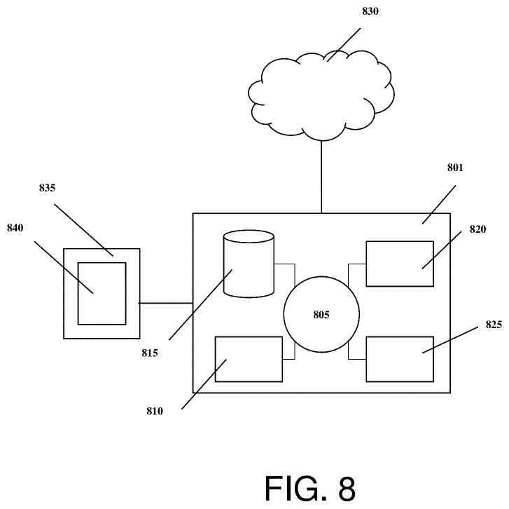

The present disclosure provides computer systems that are programmed to implement methods of the disclosure. FIG. 8 shows a computer system 801 that is programmed or otherwise configured to predict protein expressivity. The computer system 801 can regulate various aspects of expressivity prediction of the present disclosure, such as, for example, performing pre-processing and machine learning on sequence data. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a smartphone). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, results of in-silico biopanning. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, predict protein expressivity from amino acid sequence data.

### Experimental Example

The following examples are included for illustrative purposes only and are not intended to limit the scope of the invention.

### Abstract

The disclosed machine learning method may enable virtual screening of tens to thousands of candidate proteins through an “autopanning” process. Herein, “autopanning” refers to an in-silico or “virtual” biopanning technique analyzing the sequences of the protein candidates to predict expressibility of the protein candidates. The virtual biopanning technique may not require detection of peptides that bind to a select target and instead is directed towards analysis of protein candidates that can be efficiently expressed in a host cell. Results of these disclosed experiments showed 75% to 80% recall in a dataset of test proteins with nearly 100% precision for finding “high expression” proteins. Despite using relatively small training sets, these experiments quickly find the most promising candidates even when confronted with over 1,000 possible proteins or protein fragments of interest. The experiments also assessed human/machine paired techniques to accommodate a skeptical predictor. The disclosed method may accelerate discovery by allowing for more in-silico experiments prior to use of limited physical biopanning resources. Finally, the experimental results show that small increases in the training set size may relatively quickly improve model performance.

### Introduction

Finding and producing new proteins may enable the replacement of traditional animal products in more applications. Finding these new proteins may involve analyzing at a large universe of hundreds to potentially thousands of candidates for useful properties. The disclosed study considers new methods of prioritization for more efficient use of limited “biopanning” resources. Specifically, this experiment determines if in-silico experimentation can identity the proteins most likely to express well before physical experimentation on those candidates.

In-silico high-throughput screening is sometimes referred to as “virtual HTS” and may often rely on non-linear models such as random forest. Indeed, due to limited physical resources for experimentation, machine learning can use small amounts of data from actual physical (“in-vivo”) biopanning to train models which conduct larger scale simulated (“in-silico”) biopanning. In the disclosed experiment, the biopanning dataset remains relatively small but using dimensionality reduction enables less training data to be used and dimensionally-reduced phylogenetic location may be used as a proxy for strain similarity. There is limited precedent for proteome surveying using these techniques in a food space with a desire for “nature-equivalent” proteins. Therefore, this study evaluates machine learning for in-silico biopanning (“autopanning”) in the nature-equivalent food protein space.

### Dataset and Problem

Specifically, using qualitative gel analysis, modeling may use expert review of which of those biopanned proteins see high and low expression with 36 in a “high” expression category. The disclosed experiment may determine if autopanning can predict which of 114 proteins of interest (POI) across a large number of species are most likely to express well. This offers prioritization which of those proteins to next run in physical biopanning.

### Method

The disclosed experiment considered if dimensionality reduction techniques and machine learning could predict protein expression. But the phylogenetic relationship between proteins is not necessarily well understood so this experiment leveraged protein sequence similarity as a proxy. The inputs to the modelwere protein sequence (which is projected into a dimensionally reduced distance matrix) and the response is a classification of high or low expression.

### Process

This experiment first generated a fully connected (complete) graph of all proteins with nodes as proteins and edges as distances (Levenshtein) between these proteins. Then, the experiment used the Floyd-Warshall algorithm to generate a distance matrix between all proteins of interest. Next, dimensionality reduction techniques created a set of features for use in predictive modeling as described in prior work Finally, this experiment trained a series of classifiers using different techniques to use the location of a protein within the described graph through the dimensionality reduction features to predict either high or low expression where this “response” is determined by expert qualitative review of gel images.

### Modeling Approaches

This experiment considered PCA and MDS for dimensionality reduction (5 to 100 components) with model selection techniques using a form of information loss as described in other prior internal work. Using the resulting features, this study considers logistic regression (L2 regularization of 0 to 1), single decision tree (max depths from 1 to 10), random forest (up to 70 estimators with up to max depth of 10 using sqrt feature availability), and AdaBoost (up to 70 estimators with up to max depth of 10) for classifying high versus low expression. This study focused on non-neural techniques such as trees until more data become available.

### Evaluation

Given, in part, that physical experimentation guided by the “autopanner” is expected, this study forgoes a formalized test set. Therefore, this study uses 75% of the proteins with known expression in training and the remaining 25% in validation.

### Stratification

The input dataset saw class imbalance with high expression proteins making up only 32% of the dataset. This study did not force any set to have class balance but, due to the small dataset size, this study stratified such that roughly 25% of the positive cases (high expression proteins) appear in the validation set and 75% in the training set even though their assignment between the two is random.

### Results

The disclosed model achieved 100% precision and 75% recall in the validation set indicating that the autopanner can in-silico identify 75% of the actual high expressing proteins and is “right” approaching 100% of the time when given the candidate proteins' sequences.

### Dimensionality Reduction

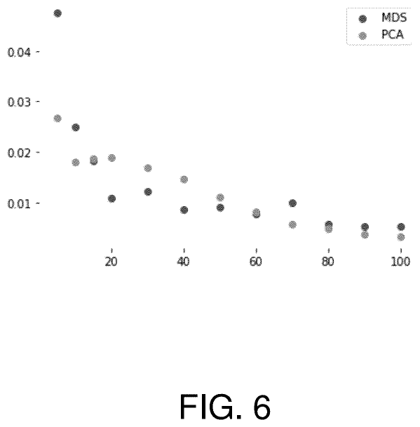

The disclosed study selected multidimensional scaling (MDS) with around 40 components. As shown in FIG. 6, use of MDS with 40 components reduced error significantly. Additionally, using 40 components for MDS compared favorably to using MDS with larger numbers of components.

During experimentation, the disclosed experiment noted that including the proteins of interest not run through in-vitro biopanning appeared to increase the number of components required from 20 to 40. This suggested that this hyperparameter selection may depend on the input proteins both in the seen and unseen sets.

### Model Training

In general, AdaBoost performed the best in the validation set by F1 score.

With the 114 in-silico only candidate proteins included in the graph, model selection chose AdaBoost at 15 estimators with max depth of 6 to achieve 100% precision and 75% recall.

### In-Silico Prediction

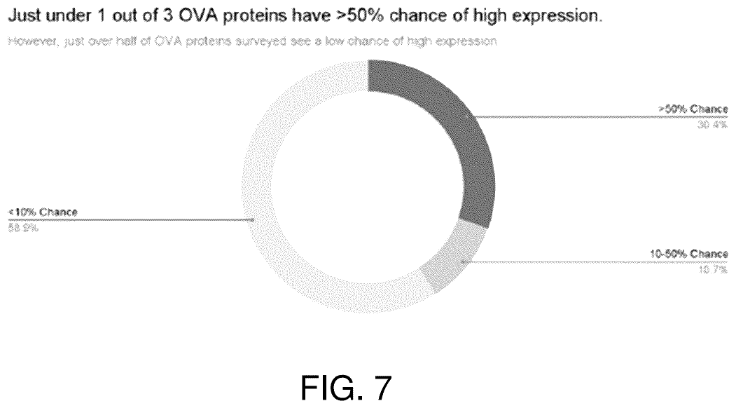

Of the 114 candidate POIs, the selected model predicted around 30% of candidate proteins see a larger than 50% chance of high expression (FIG. 7).

This set of predicted high expressing proteins included proteins from mallard, emu, saltwater crocodile, tufted duck, and burrowing owl. Notably, it also mirrored the in-vitro results of the input data in which 32% of attempted proteins saw high expression.

### Discussion

This study offered a method that, taking only the protein sequence, predicted expression.

### Larger Scale Autopanning

To test the scalability of this approach, this study also considered a protein coding gene (gg muc5b) segments experiment in which fragments of the protein run through the autopanner.

### Method

To generate segments, two options were combined. First, simulations generated fragments of variable length such that the two segments when joined create the full in order protein by sequence and, for convenience, the splits happened at every 5 amino acids. Second, simulations generated fragments of lengths 300, 500, 1000, and 1500 amino acids at every 10 amino acids.

### Results

The model selection was similar in this task to the ones described for the main experiment but 4 out of 5 of the 1,500 fragments run in that experiment reported a lower than 2.5% of high expression and none see over 50%. Still, the running time for this much larger autopanning experiment remained reasonably under 1 hour with the most time spent in dimensionality reduction, suggesting that autopanning can scale to large datasets. Note that, in this larger experiment, the model sees 100% precision and 80% recall, again highlighting that the introduction of other proteins into dimensionality reduction appears to influence model performance.

### Model Stability

Performance metrics appeared to swing depending on the dataset included in dimensionality reduction. For example, excluding the 114 “in-silico only” proteins selected AdaBoost at 10 estimators at max depth of 9 to achieve a similar F1 score but precision of 82% and recall of 90%. However, discussion saw a larger scale protein survey yielding 80% recall while sustaining the 100% precision. This may have arisen because the number of in-vitro observed proteins remained so small and this paper hypothesized that these swings would dampen with additional data.

### Accurate Precision and Recall

This experiment observed that this dataset sees class imbalance such that the validation set contains relatively few high expression proteins. This meant that the precision and recall metrics presented may be approximate such that more proteins in the validation set would give a more granular number.

### Role of Human Expertise

While posed as a classification problem, many modeling techniques including AdaBoost provided probabilities as output as well. Therefore, high value proteins may not quite get to 50% chance of high expression but researchers may still consider running them in biopanning.

### CONCLUSION

The disclosed experiment provided a method for scalable autopanning with relatively few training examples. This experiment demonstrated the ability to quickly survey a large number of candidate proteins in-silico to ensure efficient use of limited physical biopanning resources while requiring only very little data about the candidate proteins. The scale of such a fragment survey would overwhelm physical biopanning resources but use of autopanning found the relatively small number of promising candidates among hundreds, making possible an otherwise unfeasible experiment. This meant that the autopanner may have helped migrate experimentation to a post-scarcity environment where the cost of a hypothesis became low enough that the universe of hypotheses considered by human experimenters itself expanded.

### Additional Experiment

A follow-up experiment was completed that analyzed biopanning of multiple species of ovalbumin or ovalbumin-like protein.

### SUMMARY

Phylogenetic distance with MDS compression provides particularly good results with simple extraction techniques.

Future protein feature extraction techniques may include feature extraction based on graphical and statistical techniques (FEGS) and 3D-techniques, including Alphafold-esque distance matrix extraction, and potentially some protein folding extraction techniques.

### Protein Feature Extraction Results

12 simple feature extraction techniques were trialed. Well-performing strategies are below. ATC refers to atomic bond composition: the atomic bond compositions of the amino acid chains.

| | F1 | Precision | Recall |
|---|---|---|---|
| Strategy | Validation | Validation | Validation |
| Phylogenetic | 0.857 | 1 | 0.75 |
| Distance with MDS | | | |
| Amphiphilic Pseudo- | 0.875 | 0.875 | 0.875 |
| Amino Acid | | | |
| Composition | | | |
| (APAAC) no | | | |
| MDS/PCA | | | |
| ATC no MDS/PCA | 0.8 | 0.857 | 0.75 |
| (Composition of k- | 0.8 | 0.857 | 0.75 |
| spaced acid pairs) | | | |
| CKSAAP no | | | |
| MDS/PCA | | | |

### Auto-Informed Biopanning Results

In a heroic display of information-density, phylogenetic distance (with weighted Levenshtein) and MDS compression, continued to be the best representation/compression duo using an Adaboost model for prediction.

Initial biopanning results are presented below. Precision (Type I Error) is the percentage of true positives identified out of predicted positives and Recall (Type II Error) is the percentage of all true positives identified out of all actual positives.

| Summary Metrics | Test Set | Validation |
|---|---|---|
| Precision | 70% | 82% |
| Recall | 88% | 90% |
| F1 | 78% | 086% |
| Accuracy | 73% | |

### Interpreting Results

The test set used contained many more positive examples than a purely random set and they were all OVAs. This means that, in practice, this represents a good estimation of performance on a task of telling apart similar proteins. For general biopanning, the validation set performance may be more representative.

Using validation metrics, the autopanner found 90% of the high expression proteins from within a set and when it found that a protein would be be high expressing, it was right 82% of the time.

### Future Pursuits

If phylogenetic distance still stores the most information about protein expression, then there are three immediate avenues of research: deriving 3D-structure-like features from the sequences, actual 3D-structure (and information related to folding), and per colleague suggestions: folding metric feature extraction.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for identifying a subset of high-expressing proteins suitable for use in a recombinant expression system that generates recombinant proteins within a plurality of proteins, comprising:
(a) generating a distance matrix relating a plurality of amino acid sequences of a training set of amino acid sequences from a plurality of training proteins, wherein the distance matrix comprises a plurality of amino acid sequence measures of similarity;
(b) performing dimensionality reduction on a set of the plurality of amino acid sequence distances of the distance matrix to produce a set of features;
(c) generating one or more trained models by training a set of classifiers or regressors on the set of features; and
(d) using the one or more trained models, analyzing a test set of amino acid sequences from the plurality of proteins, to identify whether the subset of proteins within the plurality of proteins comprises high-expressing proteins, wherein analyzing the test set of amino acid sequences comprises:
(i) repeating steps (a) and (b) for the test set of amino acid sequences to produce a set of test features; and
(ii) processing the set of features with the one or more trained models to predict an expressivity of one or more proteins corresponding with one or more amino acid sequences from the test set of amino acid sequences.

2. The method of claim 1, wherein an amino acid sequence corresponds to a protein or to a fragment of a protein.

3. The method of claim 1, wherein the plurality of proteins includes comprises none of the plurality of training proteins.

4. The method of claim 1, wherein the distance matrix is created at least in part by calculating a Levenshtein distance between at least one pair of the plurality of amino acid sequences.

5. The method of claim 1, wherein the distance matrix is calculated at least in part using a Floyd-Warshall algorithm.

6. The method of claim 1, wherein the dimensionality reduction is principal component analysis (PCA) or multidimensional scaling (MDS).

7. The method of claim 6, wherein the multidimensional scaling uses from about 20 to about 40 components.

8. The method of claim 1, wherein the set of classifiers comprises at least one of a logistic regression, a decision tree, gradient boosted trees, a random forest model, neural network or Adaboost.

9. The method of claim 1, wherein the set of features comprises no more than 20 features.

10. The method of claim 9, wherein the set of features comprises from about 10 to about 20 features.

11. The method of claim 1, wherein a protein of the set of high-expressing proteins is expressed in a cell.

12. The method of claim 11, wherein the cell is a recombinant cell.

13. The method of claim 12, wherein the recombinant cell is a microbial cell.

14. The method of claim 13, wherein the microbial cell is from a microbial organism selected from a Komagataella species, a Saccharomyces species, a Trichoderma species, a Pseudomonas species, an Aspergillus species, and an E. coli species.

15. The method of claim 1, wherein the plurality of proteins comprises one or more animal or egg proteins.

16. The method of claim 15, wherein the one or more egg proteins are selected from ovalbumin (OVA), ovomucoid (OVD), ovotransferrin, lysozyme proteins, ovomucin, ovoglobulin G2, ovoglobulin G3, ovoinhibitor, ovoglycoprotein, flavoprotein, ovomacroglobulin, ovostatin, cystatin, avidin, ovalbumin related protein X, and ovalbumin related protein Y, and any combination thereof.

17. The method of claim 16, wherein the one or more egg proteins are naturally expressed by poultry, fowl, waterfowl, game bird, chicken, quail, turkey, turkey vulture, hummingbird, duck, ostrich, goose, gull, guineafowl, pheasant, emu, crocodile, owl, finch, pigeon, penguin, or any combination thereof.
